(19) United States
(12) Patent Application Publication
(10) Pub. No.: US 2022/0230494 A1
(43) Pub. Date: Jul. 21, 2022

(54) SECURITY SYSTEMS AND PROCESSES INVOLVING BIOMETRIC AUTHENTICATION

(71) Applicant: Active Witness Corp., White Rock (CA)

(72) Inventors: Rajeev Kumar Bakshi, Surrey (CA); David Allan Black, North Vancouver (CA); Joseph P, Weil, Highland Park, IL (US)

(21) Appl. No.: 17/606,722

(22) PCT Filed: Apr. 29, 2020
(86) PCT No.: PCT/CA2020/050567

§ 371 Date: Oct. 26, 2021

Related U.S. Application Data
(60) Provisional application No. 62/839,968, filed on Apr. 29, 2019.
(60) Provisional application No. 62/893,368, filed on Aug. 29, 2019.
(60) Provisional application No. 63/009,381, filed on Apr. 13, 2020.

(51) Int. Cl. G07C 9/00; H04L 9/40; G06K 7/14; G06V 40/16

(52) U.S. Cl. CPC G07C 9/00563; G07C 9/00571; H04L 63/0807; G06V 40/168; G06K 7/1417; G06V 40/161; G06K 7/1413

(57) ABSTRACT

Various embodiments for an automatic security system to allow a person to access a space upon authentication are provided. Various embodiments for methods of unlocking of an electronic access control device of a security system are also provided. The system and method involve the performance of a two-step authentication in which at least one step involves the evaluation of facial features, including a step which involves the presentation of adjusted facial features. The system and methods may be used to prevent or limit persons accessing a space they are not authorized to access.

### RELATED APPLICATION

This application claims the benefit of U.S. Provisional Patent Application No. 62/839,968 filed Apr. 29, 2019; U.S. Provisional Patent Application No. 62/893,368, filed on Aug. 29, 2019; and U.S. Provisional Patent Application No. 63/009,381, filed on Apr. 13, 2020; the entire contents of Patent Applications 62/839,968; 62/893,368 and 63/009,381 are hereby incorporated by reference.

### BACKGROUND OF THE DISCLOSURE

The present disclosure relates to security systems and processes, and more in particular, to security systems and processes involving biometric authentication.

### FIELD OF THE DISCLOSURE

The following paragraphs are provided by way of background to the present disclosure. They are not, however, an admission that anything discussed therein is prior art or part of the knowledge of persons skilled in the art.

Many security systems have evolved to control access to spaces containing valuable assets or resources. Such spaces include physical spaces, such as home and work spaces, and electronic spaces containing valuable information, such as an automated teller machine (ATM). Comparatively, recent security systems have come to include automated authentication systems, which require limited or no direct interaction between a person seeking access to a secured space and a person responsible for controlling such access. These automated authentication systems are generally deemed desirable, as they can reduce or exclude human error or weaknesses, and limit the costs associated with the operation of the security system.

However, a significant inherent technical challenge associated with automatic authentication systems consists of the design and configuration of system components allowing an authentication system to grant access to an individual legitimately presenting himself or herself to the system, and, conversely, to deny access to an individual who illegitimately presents himself or herself to the authentication system. Authentication systems that rely on unique and unalterable biometric features, such as fingerprints or facial features, in this regard, are considered to be strong authentication systems. Nevertheless unscrupulous individuals have been known to circumvent even these biometric authentication systems, for example, by presenting a facial image or a video to the camera of an authentication system. It will be clear that the assets' owners can be duped by those having illegitimately obtained access thereto.

A further challenge with known automated facial recognition systems is that when datastores comprise the facial features of a large number of individuals, it becomes more challenging and/or more time consuming for automated authentication systems to correctly distinguish persons with similar facial features.

Therefore, there exists in the art a need for improved automated security systems and processes for controlling access to valuable assets and resources. An automated system that is capable of rapid and accurate authentication based on unique personal features, which can readily be implemented to control access to a wide variety of resources and assets is particularly desirable.

### SUMMARY OF THE DISCLOSURE

The following paragraphs are intended to introduce the reader to the more detailed description that follows and not to define or limit the claimed subject matter of the present disclosure.

In one broad aspect, the present disclosure relates to a security system involving facial biometric authentication. Accordingly, the present disclosure provides, in at least one aspect, in at least one embodiment, a security system comprising:
an electronically lockable access control device configured to be unlocked upon authentication of a person presenting to the access control device; and
an authentication module coupled to the access control device, the authentication module comprising:
   an instruction device;
   a camera configured to capture a first facial image of at least a portion of the face of the person presenting to the camera; and
   a central controller comprising a processor and a memory that is accessible by the processor, the central controller being communicatively coupled to the instruction device and the camera, and the memory having program instructions stored thereon, that when executed by the processor, configure the central controller to:
      perform a first authentication step of a two-step authentication process on the person, the first authentication step comprising:
         receiving a first authentication token from the person and authenticating the first authentication token; and
      perform a second authentication step of the two-step authentication process on the person, the second authentication step comprising:
         selecting one of a plurality of facial adjustment instructions to instruct the person to adjust at least one facial feature when being imaged by the camera;
         sending the selected facial adjustment instruction to the instruction device;
         providing the selected facial adjustment instruction via the instruction device to the person;
         capturing, via the camera, a second facial image of the person while the person is adjusting the at least one facial feature in accordance with the transmitted facial adjustment instruction;
         receiving, at the central controller, at least a portion of the second facial image comprising the at least one adjusted facial feature of the person; and
         authenticating the person when the portion of the second facial image is matched with a corresponding stored authorized adjusted facial image of the person obtained from a datastore of adjusted facial images of the person; and
      unlock the access control device when there is successful authentication in the first and second authentication steps.

In at least one embodiment, the second authentication step can be performed only when there is successful authentication in the first step.

In at least one embodiment, the camera is configured to capture and receive the first authentication token.

In at least one embodiment, the authentication module can comprise an additional device that is configured to receive the first authentication token wherein the additional device is a device other than the camera.

In at least one embodiment, the central controller can be in communication with a datastore comprising a plurality of stored authorized authentication tokens and the first authentication step comprises performing a matching between the received authentication token and the stored authorized authentication tokens, where each stored authorized authentication token is linked to stored authorized facial images comprising adjusted facial features of the person, and the central controller is configured to perform the authenticating in the second authentication step by performing solely a matching between the captured adjusted facial image and one of the stored authorized facial images that are linked to the first authentication token and comprise adjusted facial features of the person.

In at least one embodiment, the central controller is configured to search the stored authorized facial images that have one or more facial adjustments that correspond with one or more facial adjustments in the provided facial adjustment instruction.

In at least one embodiment, the first authentication token can comprise a 1D or 2D barcode.

In at least one embodiment, the first authentication token can comprise the first facial image captured by the camera, and the authentication can comprise performing a matching between the captured first facial image against a datastore comprising stored authorized facial images.

In at least one embodiment, the camera or the instruction device can be situated in close proximity to the electronically lockable access control device.

In at least one embodiment, the instruction device can be configured to provide visual instructions or audible instructions to the person.

In at least one embodiment, the visual instructions can comprise a cartoon representing an adjusted facial feature.

In at least one embodiment, the visual instructions can comprise text instructions for the person to adjust at least one of their facial features.

In at least one embodiment, the central controller can be configured to perform the first and the second authentication step in different first and second spaces, respectively.

In at least one embodiment, the electronic access control device can comprise first and second electronic access control components, the first electronic access control component being unlocked upon successful authentication in the first authentication step, and the second electronic access control component being unlocked upon successful authentication in the second authentication step.

In at least one embodiment, the central controller can be configured to unlock the access control device only when the first and/or second authorization step is also performed at a selected pre-approved time.

In at least one embodiment, the electronic access control device can further include a temperature-detection device to detect the body temperature of the person, the temperature-detection device being coupled to the central controller, the central controller being configured to unlock the access control device when the detected body temperature of the person is within a predefined body temperature range.

In at least one embodiment, the temperature-detection device can be configured to detect the body temperature of the person following the performance of the first and second authentication step.

In at least one embodiment, the predefined body temperature can range from about 36.5° C. to about 38.5° C.

In another aspect, the present disclosure relates to methods for unlocking of an electronic access control device of a security system comprising a central controller. Accordingly, the present disclosure provides in at least one aspect, a computer implemented method for unlocking of an electronic access control device of a security system, the method comprising:
capturing, via a camera, a facial image of a person presented to the camera where the camera is positioned in proximity of the electronic access control device;
performing a first authentication step of a two-step authentication process on the person, the first authentication step comprising:
   receiving a first authentication token; and authenticating the presented person using the first authentication token;
performing a second authentication step of the two-step authentication process on the person, the second authentication step comprising:
   selecting one of a plurality of facial adjustment instructions to instruct the person to adjust at least one facial feature when being imaged by the camera;
   prompting an instruction device to transmit the selected facial adjustment instruction to the person;
   capturing, via the camera, a facial image of the person adjusting the at least one facial feature in accordance with the transmitted facial adjustment instruction;
   receiving at least a portion of the second facial image comprising the adjusted facial feature; and authenticating the person when the portion of the
   facial image is matched with a corresponding stored image portion of the person from a datastore of adjusted facial images; and
unlocking the access control device upon successful authentication of the person in the first and second authentication steps.

In at least one embodiment, the method comprises performing the second authentication step only when there is successful authentication in the first step.

In at least one embodiment, the method comprises using the camera to capture and receive the first authentication token.

In at least one embodiment, the method comprises performing the first authentication step using an additional device that is configured to receive the first authentication token wherein the additional device is a device other than the camera.

In at least one embodiment, the first authentication step can comprise performing a matching between the received authentication token and stored authorized authentication tokens, where each stored authorized authentication token is linked to stored facial images comprising adjusted facial features of the person, and the authenticating in the second authentication step is performed solely based on a matching between the captured adjusted facial image and one of the stored authorized facial images that are linked to the first authentication token and comprise adjusted facial features of the person.

In at least one embodiment, the method comprises searching the stored authorized facial images that have one or more facial adjustments that correspond with one or more facial adjustments in the provided facial adjustment instruction.

In at least one embodiment, the first authentication token can comprise a 1D or 2D barcode.

In at least one embodiment, the first authentication token can comprise the first facial image captured by the camera, and the authentication can comprise performing a matching between the captured first facial image against a datastore comprising stored authorized facial images.

In at least one embodiment, the camera or the instruction device can be situated in close proximity to the electronically lockable access control device.

In at least one embodiment, wherein the method comprises using the instruction device to provide visual instructions or audible instructions to the person.

In at least one embodiment, wherein the visual instructions can comprise a cartoon representing an adjusted facial feature.

In at least one embodiment, the visual instructions can comprise text instructions for the person to adjust at least one of their facial features.

In at least one embodiment, the first and the second authentication step can be performed in different first and second spaces, respectively.

In at least one embodiment, the electronic access control device can comprise first and second electronic access control components, and the method comprises unlocking the first electronic access control component upon successful authentication in the first authentication step, and unlocking the second electronic access control component upon successful authentication in the second authentication step.

In at least one embodiment, the method comprises unlocking the access control device only when the first and/or second authorization step is also performed at a selected pre-approved time.

In at least one embodiment, the electronic access control device can further include a temperature-detection device and the method further comprises detecting the body temperature of the person with the temperature-detection device and unlocking the access control device when the detected body temperature of the person is within a predefined body temperature range.

In at least one embodiment, wherein the method comprises using the temperature-detection device to detect the body temperature of the person following the performance of the first and second authentication step.

In at least one embodiment, the predefined body temperature range can range from about 36.5° C. to about 38.5° C.

Other features and advantages of the present disclosure will become apparent from the following detailed description. It should be understood, however, that the detailed description, while indicating some implementations of the disclosure, are given by way of illustration only, since various changes and modifications within the spirit and scope of the disclosure will become apparent to those of skill in the art from the detailed description.

### BRIEF DESCRIPTION OF THE DRAWINGS

The disclosure is in the hereinafter provided paragraphs described, by way of example, in relation to the attached figures. The figures provided herein are provided for a better understanding of the example embodiments and to show more clearly how the various embodiments may be carried into effect. The figures are not intended to limit the present disclosure.

FIG. 1 is a schematic view of a security system comprising an electronic access control device according to an example embodiment of the present disclosure.

FIG. 2 is a schematic view of an electronically lockable access control device according to an example embodiment of the present disclosure.

FIGS. 3A-3B illustrate a facial image (FIG. 3A) and a facial image comprising adjusted facial features (FIG. 3B) according to an example embodiment of the present disclosure.

FIG. 4 is a schematic view of authentication tokens that are stored in a datastore according to an aspect of an example embodiment of the present disclosure.

FIG. 5 is a flow chart of a method for unlocking of an electronically lockable access control device of a security system according to an example embodiment of the present disclosure.

FIG. 6 is another example embodiment of a security system comprising an electronic access control device in accordance with the teachings herein.

### DETAILED DESCRIPTION

Various systems and processes will be described below to provide an example of an implementation or embodiment of each claimed subject matter. No implementation or embodiment described below limits any claimed subject matter and any claimed subject matter may cover methods, systems, devices, assemblies, processes or apparatuses that differ from those described below. The claimed subject matter is not limited to systems or processes having all of the features of any one system, method, device, apparatus, assembly or process described below or to features common to multiple or all of the systems, methods, devices, apparatuses, assemblies or processes described below. It is possible that a system or process described below is not an implementation or embodiment of any claimed subject matter. Any subject matter disclosed in a system or process described below that is not claimed in this document may be the subject matter of another protective instrument, for example, a continuing patent application, and the applicants, inventors or owners do not intend to abandon, disclaim or dedicate to the public any such subject matter by its disclosure in this document.

As used herein and in the claims, the singular forms, such as “a”, “an” and “the” include the plural reference and vice versa unless the context clearly indicates otherwise. Throughout this specification, unless otherwise indicated, the terms “comprise,” “comprises” and “comprising” are used inclusively rather than exclusively, so that a stated integer or group of integers may include one or more other non-stated integers or groups of integers.

The term “or” is inclusive unless modified, for example, by “either”.

When ranges are used herein, such as for geometric parameters, for example distances, all combinations and sub-combinations of ranges and specific implementations therein are intended to be included. Other than in the operating examples, or where otherwise indicated, all numbers expressing quantities of ingredients or reaction conditions used herein should be understood as being modified in all instances by the term “about.” The term “about” when referring to a number or a numerical range means that the number or numerical range being referred to is an approximation within experimental variability (or within statistical experimental error), and thus the number or numerical range may vary between 1% and 15% of the stated number or numerical range, as will be readily recognized by context. Furthermore any range of values described herein is intended to specifically include the limiting values of the range, and any intermediate value or sub-range within the given range, and all such intermediate values and sub-ranges are individually and specifically disclosed (e.g. a range of 1 to 5 includes 1, 1.5, 2, 2.75, 3, 3.90, 4, and 5). Similarly, other terms of degree such as “substantially” and “approximately” as used herein to modify a term is understood to mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular implementations only, and is not intended to limit the scope of the present disclosure, which is defined solely by the claims.

All publications, patents and patent applications are herein incorporated by reference in their entirety to the same extent as if each individual publication, patent or patent application was specifically and individually indicated to be incorporated by reference in its entirety.

### Definitions

The terms “automated system” or “system”, as used interchangeably herein, refers to a device, or configuration of a plurality of devices, with one or more electronic processing elements capable of performing machine executable program instructions where the devices include but are not limited to, any personal computer, desktop computer, hand-held computer, laptop computer, tablet computer, cell phone computer, smart phone computer or other suitable electronic device or plurality of devices.

A portion of the example embodiments of the systems, devices, or methods described in accordance with the teachings herein may be implemented as a combination of hardware or software. For example, a portion of the embodiments described herein may be implemented, at least in part, by using one or more computer programs, executing on one or more programmable devices each comprising at least one processing element, and at least one data storage element (including volatile and non-volatile memory). These devices may also have at least one input device and at least one output device as defined herein.

It should also be noted that there may be some elements that are used to implement at least part of the embodiments described herein that may be implemented via software that is written in a high-level procedural language such as object-oriented programming. The program code may be written in MATLAB™, Visual Basic, Fortran, C, C++ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media (e.g., a computer readable medium such as, but not limited to, ROM, magnetic disk, optical disc) or a device that is readable by a general or special purpose programmable device. The software program code, when read by at least one processor of the programmable device, configures the at least one processor to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable/readable instructions, such as program code or program instructions, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, USB keys, external hard drives, magnetic and electronic media storage, tablet (e.g. iPad) or smartphone (e.g. iPhones) apps, and the like, for example. In alternative embodiments, the medium may be transitory in nature such as, but not limited to, wire-line transmissions, satellite transmissions, internet transmissions (e.g. downloads), media, as well as digital and analog signals, for example. The computer useable instructions may also be in various formats, including compiled and non-compiled code.

The term “coupled”, as used herein, can have several different meanings depending on the context in which the term is used. For example, the term coupled can have a mechanical or electrical connotation depending on the context in which it is used, i.e. whether describing a physical layout or transmission of data as the case may be. For example, depending on the context, the term coupled may indicate that two elements or devices can be directly physically or electrically connected to one another or connected to one another through one or more intermediate elements or devices via a physical or electrical element such as, but not limited to, a wire, a non-active circuit element (e.g. resistor) and the like, for example.

The term “input device”, as used herein, refers to any user operable device that is used to input information and includes but is not limited to, one or more of a terminal, a touch screen, a keyboard, a mouse, a mouse pad, a tracker ball, a joystick, a microphone, a voice recognition system, a light pen, a camera, a data entry device, such as a barcode reader or a magnetic ink character recognition device, a sensor or any other computing unit capable of receiving input data. In some embodiments, input devices may comprise a two dimensional display, such as a TV or a liquid crystal display (LCD), a light-emitting diode (LED) backlit display, or a mobile telephone display capable of receiving input from a user e.g. by touch screen. The user in accordance herewith may be any user or operator including, for example any safety manager, or work site operator or manager.

The term “output device”, as used herein, refers to any device that is used to output information and includes, but is not limited to, one or more of a display terminal, a screen, a printer (e.g. laser, inkjet, dot matrix), a plotter or other hard copy output device, speaker, headphones, electronic storage device, a radio or other communication device, that may communicate with another device, or any other computing unit. Output devices may also comprise a two dimensional display, such as a television or a liquid crystal display (LCD), a light-emitting diode (LED) backlit display, and/or a mobile telephone display, capable of providing output data in a user viewable format.

### General Implementation of the System

As hereinbefore mentioned, the present disclosure relates to automated security systems and processes involving biometric authentication. The automated security system and processes can be implemented in a manner that controls access to valuable resources via a lockable access control device, in such a manner that only persons having been authorized can gain access via the lockable access control device. In particular, the system and processes of the present disclosure involve authentication based on facial biometric information. The system can be configured to identify imposters or hackers presenting copies of authenticated facial images, for example, based on photographs thereof, and can deny access to such imposters or hackers. Furthermore, the present system can be configured to limit the amount of computing operations required to perform a facial recognition step and/or limit errors as a result of persons presenting for authentication with similar facial features. These and other beneficial aspects, render the herein disclosed system useful in safeguarding valuable resources from unauthorized access thereto.

Accordingly, the present disclosure provides, in at least one aspect, at least one embodiment of a security system comprising:
an electronically lockable access control device configured to be unlocked upon authentication of a person presenting to the access control device; and
an authentication module coupled to the access control device, the authentication module comprising:
an instruction device;
a camera configured to capture at least a portion of a facial image of the person presenting to the camera; and
a central controller comprising a processor and a memory that is accessible by the processor, the central controller being communicatively coupled to the instruction device and the camera, and the memory having program instructions stored thereon, that when executed by the processor, configure the central controller to:
   perform a first authentication step of a two-step authentication process on the person, the first authentication step comprising:
      receiving a first authentication token and authenticating the first authentication token; and
   perform a second authentication step of the two-step authentication process on the person, the second authentication step comprising:
      selecting one of a plurality of facial adjustment instructions to instruct the person to adjust at least one facial feature when being imaged by the camera;
      sending the selected facial adjustment instruction to the instruction device;
      transmitting the selected facial adjustment instruction via the instruction device to the person;
      capturing, via the camera, a facial image of the person adjusting the at least one facial feature in accordance with the transmitted facial adjustment instruction;
      receiving, at the central controller, at least a portion of the facial image comprising the at least one adjusted facial feature of the person; and
      authenticating the person when the portion of the facial image is matched with a corresponding stored image portion of the person from a datastore of adjusted facial images of the person; and
   unlock the access control device when there is successful authentication in the first and second authentication steps.

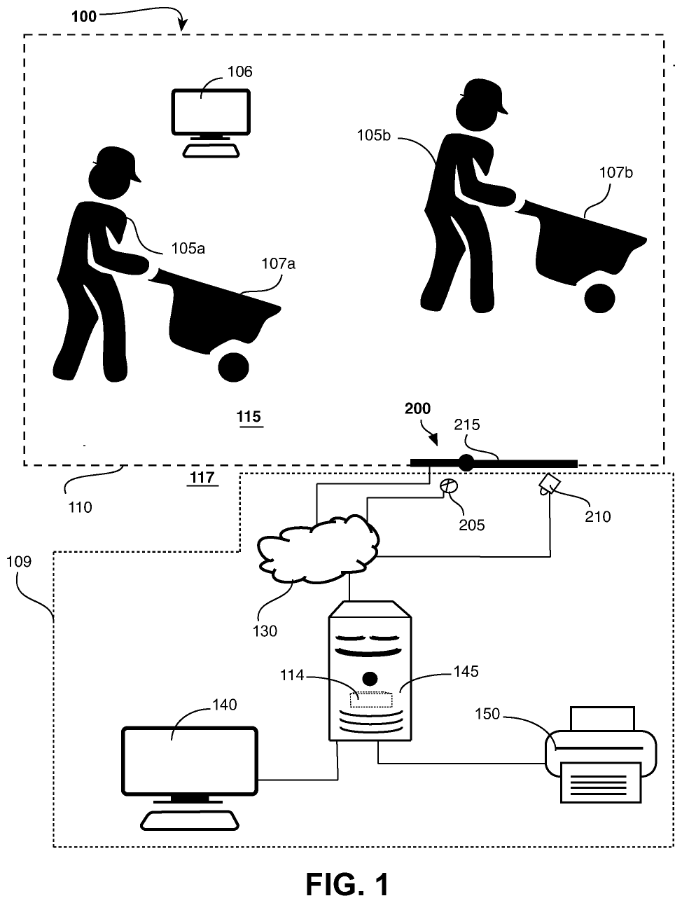

An example embodiment of a security system according to the present disclosure is shown in FIG. 1. Thus, referring now to FIG. 1, the present disclosure provides, in an example embodiment, a security system 100 to limit access to work space 115, separated by fence 110 or other enclosure from exterior space 117. Work space 115 contains certain operational assets, namely wheel barrows 107a and 107b and computing device 106. It is noted that the space, represented by way of example as work space 115, and assets, represented by way of example as wheel barrows 107a and 107b and computing device 106, according to different embodiments of the present disclosure, can be any space to which one wishes to control access, including any work space or any private space, including a private home space. Furthermore, the assets can be any physical asset, such as equipment, documents, or monetary currency, for example. The assets can also be electronic information, such as personal information, banking information, electronic user profile information, etc. Work space 115 can be a physical space, such as, but not limited to, a building or terrain, for example, which can be separated from exterior space 117 by any access-limiting structure defining the perimeter of the space, such as a wall, fence, barricade, railing, hedge, or any other barrier structure. In other embodiments, work space 115 can also be a virtual space, for example, an electronic domain or space containing information, accessible via an input device, such as a computer terminal. Thus, for example, work space 115, in some embodiments, can be a computer domain comprising electronic banking information of a person, to which a person seeks access via an automatic teller machine (ATM). It is to be expressly understood that the present disclosure is not limited by the particular space, work space or private space, or the particular assets contained therein and accessible using the security system of the present disclosure. The security system of the present disclosure may be implemented in conjunction with any space and any assets contained therein.

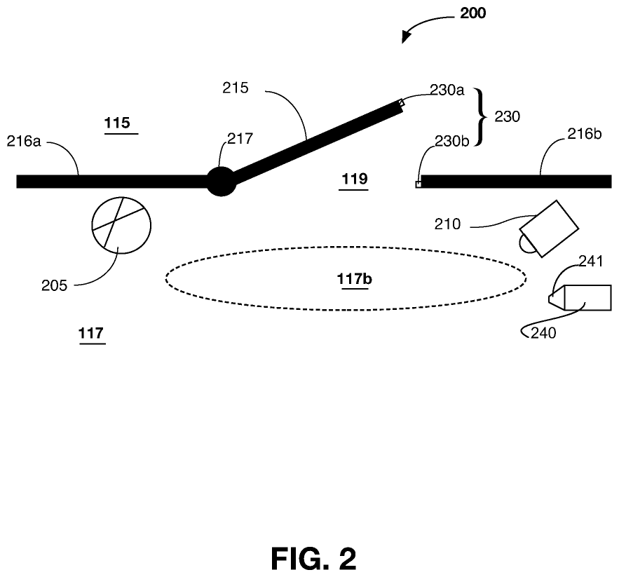

Still referring to FIG. 1, persons 105a and 105b have gained access to work space 115 from exterior space 117 by electronically unlocking openable gate 215. In this respect, openable gate 215 is part of electronically lockable access control device 200. Locking and unlocking of openable gate 215 is controlled by an authentication module 109 comprising central controller 145, camera 205 and instruction device 210, as hereinafter further described with reference to FIG. 2. Central controller 145 is a server containing a processor and a memory having program instructions stored thereon. The central controller 145 is electronically coupled to electronically lockable access control device 200 via network 130. Authentication module 109 further comprises input device 140 and output device 150, each of which are coupled to central controller 145 to, respectively, permit input to central controller 145 and in this manner operate central controller 145, and receive output from central controller 145, as may be desired by an operator of central controller 145.

Central controller 145 comprises any suitable computer processor that can provide sufficient processing power depending on the requirements of central controller 145 as is known by those skilled in the art. Central controller 145 may include one processor. Alternatively, there may be a plurality of processors that are used by central controller 145, and these processors may function in parallel and perform certain functions. In alternative embodiments, specialized hardware can be used to provide some of the functions provided by the central controller 145.

Central controller 145 can include ports and/or devices that allows central controller 145 to communicate with other devices or computers. In some cases, these can include at least one of a serial port, a parallel port or a Universal Serial Bus (USB) port that provides USB connectivity. Central controller 145 can also include at least one of an Internet, Local Area Network (LAN), Ethernet, Firewire, modem or digital subscriber line connection. For example, central controller 145 can include a standard network adapter such as an Ethernet or 802.11x adapter. In some embodiments, central controller 145 may include a radio that communicates utilizing CDMA, GSM, GPRS or Bluetooth protocol according to standards such as IEEE 802.11a, 802.11b, 802.11g, or 802.11n. Various combinations of these elements can be incorporated within or used by central controller 145.

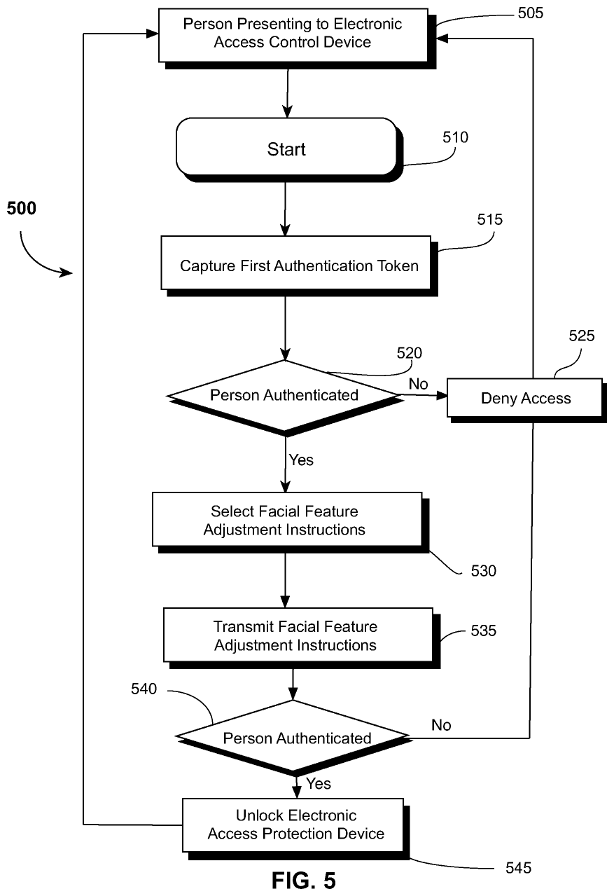

Datastore 114 that is included in the central controller 145 can include RAM, ROM, one or more hard drives, one or more flash drives or some other suitable data storage elements such as disk drives, etc. Datastore 114 may store the program instructions for an operating system, program code for various applications, and one or more databases. The programs comprise program code that, when executed, configures central controller 145 to operate in a particular manner to implement various functions, tools, processes, and methods for the security system 100. For example, the program code may include software instructions for performing various methods in accordance with the teachings herein examples of which are shown in FIG. 5. Datastore 114 may also store various operational parameters, authentication tokens, and/or authentication results. In some embodiments, datastore 114 may be a separate device that is remotely accessible by the central controller 145, in which case certain elements that were described previously as being stored in the datastore 114 may be alternatively or additionally stored in the memory of the central controller 145.

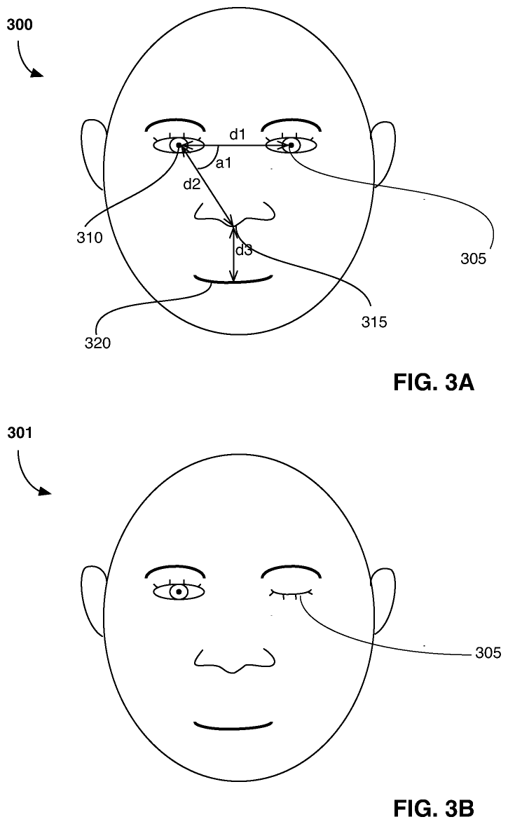

Referring now to FIGS. 2 and 3A-3B, shown in FIG. 2 is electronically lockable access control device 200, situated at the perimeter of work space 115, and separating work space 115 from exterior space 117. Electronically lockable access control device 200 comprises openable gate 215 having a rotatable gate hinge element 217, and gate support structures 216a, 216b. Electronically lockable access control device 200, as noted, is coupled to central control unit 145 via network 130. In order to transition openable gate 215 from a locked and closed position as shown in FIG. 1 to the opened position, as shown in FIG. 2, central controller 145 can transmit a signal to access control device 200 to release electronically interlocking elements 230a and 230b, which can, for example, be electromagnetic interlocking elements, together forming electronic lock 230, thus allowing openable gate 215 to be opened or closed.

Security system 100 is further configured so that upon successful authentication of a person wishing to access work space 115 from exterior space 117 via passage way 119, openable gate 215 is unlocked. When openable gate 215 is opened from a closed position (shown in FIG. 1) passage way 119 is formed, as can be seen in FIG. 2. Thus, in other words, security system 100 is configured to require authentication of a person wishing to access work space 115 from exterior space 117 prior to providing access to work space 115. In this manner, access to work space 115 can be controlled and limited by an owner or operator of security system 100. This includes providing access to work space 115 to certain persons, and not others, and providing access to certain persons to work space 115 for a certain period of time and not others, for example, access to work space 115 for a selected week, or daytime access to work space 115 only, as hereinafter further described.

Security system 100 is further configured to perform a two-step authentication process. Initially a person wishing to access work space 115 approaches openable gate 215 which is closed in a locked position. The person then presents a first authentication token. “Authentication token” as used herein refers to a physical object that contains any collection of features, including biometric features, and is contained by any medium that is receivable by a security system for the purpose of identifying the presenting person. The first authentication token in some embodiments is an identification card, for example, an identification card containing a 1-dimensional (1D) linear barcode, or a two-dimensional (2D) barcode, e.g. a QR code, DataMatrix or PDF417. Such bar codes may optionally include error correcting codes such as forward error correction (FEC) based codes, or Reed-Solomon based codes, for example.

Referring again to FIGS. 1 and 2, all or some of the information on the first authentication token is presented and captured by camera 205 installed in the proximity of openable gate 215. It is noted in this respect that camera 205 is preferably installed within meters, or tens of meters, from openable gate 215. In some embodiments, camera 205, as well as instruction device 210 (which, as hereinafter discussed, is also installed in the proximity of openable gate 215) are integrated with the structure comprising openable gate 215, for example, within or attached to gate support structures 216a, 216b. In other embodiments, another camera or other device capable of receiving the first authentication token, for example, a scanner, placed within the proximity of openable gate 215 may be used to present the first authentication token. As will be clear, the device used to receive the first authentication token is selected to be compatible with the format of the first authentication token, i.e. the device is configured to be able to obtain the relevant information for authentication purposes from the first authentication token, and may vary depending on the physical nature of the selected first authentication token (e.g. whether the token is an ID card with a barcode on its surface or an ID card that has an embedded chip which contains barcode, or other identification data). The device thus may be a scanner, a chip reader, a camera, or the like selected to match the format of the first authentication token for acquisition thereof.

In at least one embodiment, the first authentication token comprises biometric features, including, for example, fingerprints or facial biometric features. Such biometric features may be captured in the form of a visual image, for example a facial image, of the person who is carrying the first authentication token. In this respect, the term “facial image” means an image of the entirety of person's face or of a portion of a person's face. Referring again to FIGS. 1 and 2, a facial image may be captured by camera 205 after the person has situated themselves within presentation space 117b. This facial image can be referred to as the first authentication token. It is noted that in some embodiments, the person may initiate the first authentication step by performing an action, for example, by pressing an initiating button coupled to camera 205, and, for example, installed in close proximity of camera 205, to thereby prompt camera 205 to capture a facial image of the presenting person. In other embodiments, camera 205 may include a sensor capable of detecting when a person moves within presentation space 117b, and camera 205 may automatically capture a facial image upon detection of the person in presentation space 117b. In this case, the captured facial image is the first authentication token.

Camera 205 transmits the captured first authentication token to central controller 145. Central controller 145 is configured to access stored authentication tokens of all persons authorized access work space 115 within a datastore 114 of a memory component. Stored authentication tokens may be entered for storage in datastore 114 of the memory component of central controller 145 via input device 140, operated, for example, by a human administrative operator of security system 100. Upon such entry of these authentication tokens in datastore 114, the authorization tokens become stored authorized authentication tokens. In this manner datastore 114 can be configured to comprise a plurality of stored authorized authentication tokens, and may include, for example, hundreds, thousands, tens of thousands, or more stored authorized authentication tokens. Thus, referring again to FIG. 1, it will be understood that separate authentication tokens for workers 105a and 105b may be stored in datastore 114. Within database 114 stored authentication tokens are generally preferably linked to personal information, for example names, birth dates, telephone, and so on, of workers 105a and 105b. This allows the owner or operator of security system 100 to identify worker 105a and 105b, for example, in the event access to space 115 is denied, and worker 105a or 105b, or the owner or operator of security system 100 wish to investigate the cause of the denied access.

To perform the first authentication step, central controller 145 is configured to compare the captured first authentication token from the person who is presenting themselves at the presentation space 117b with the stored authorized authentication tokens in datastore 114. In this respect, depending on the format of the captured first authentication token, various features of the first authentication token may be compared with those present in datastore 114. For example, in embodiments hereof where 1D or 2D barcodes are used as authentication tokens, features of the presented barcodes, including visual patterns (e.g. for 1D barcodes: number of bars, size of bars, relative distance between bars) are compared with features of barcodes of the stored authorized authentication tokens. Central controller 145 is configured to identify a barcode in a stored authorized authentication token in datastore 114 with identical features to those of the presented barcodes and thus establish a match between the two barcodes. Machine executable program code to configure central controller 145 in this respect is well known to those of skill in the art and includes, for example, Google® ZXing barcode scanning software (http://code.google.com/p/zxing/), Apple® Scan for iPhone, Optiscan, QRafter, ScanLife, I-Nigma, Quickmark, Kaywa Reader, Nokia® Barcode Reader, Blackberry® Messenger, Esponce® QR Reader, and/or the like.

In embodiments hereof where the first authentication token comprises a captured facial image, or a portion thereof, the first authentication step comprises identifying a possible match between the captured facial image and the stored authorized authentication tokens, including facial images that are stored in datastore 114, based on distinct facial features. Such a match may be based on facial geometry, for example, as illustrated in FIG. 3A, which may include, but is not limited to, one of the following measures: the interpupil distance d1 between right eye pupil 310 and left eye pupil 305, the distance d2 between right eye pupil 310 and nose tip 315, the distance d3 between nose tip 315 and lips 320, and angle a1 defined by d1 and d2 represent example geometries defining facial features, for example. Other suitable methods and techniques which allow identification based on a match between presented facial features in a captured image and facial features in a stored image are known to the art, and include, for example, the methods and techniques described in U.S. Pat. No. 8,406,484,which is included herein by reference. Furthermore, neural network based pattern matching of facial characteristics may be used either alone, or in combination with facial geometry based facial geometry based matching (see: e.g. U.S. Pat. No. 10,333,714, which is included herein by reference).

In the absence of a match between the stored authorized authentication tokens and the first authentication token access is denied and openable gate 215 remains closed in a locked position. If, on the other hand, a match is established between a stored authorized authentication token and the first authentication token, central controller 145 performs a second authentication step which includes transmitting a facial adjustment instruction to instruction device 210, which in turn transmits the facial adjustment instruction to the presenting person.

Instruction device 210, which like camera 205 is installed in proximity of openable gate 215, can be any device capable of transmitting a facial adjustment instruction to the presenting person, including a visual or audible instruction, and includes for example a two-dimensional display, an LCD display, for example, or an audio speaker. Visual instructions include text based instructions or image based instructions, e.g. a cartoon instruction, such as shown in FIG. 3B, which is an image that is used to instruct person 301 to close his left eye 305. Such a facial adjustment instruction is selected, preferably randomly, from multiple possible facial adjustment instructions to adjust one or more facial features of the presenting person during the second authentication step. These include, for example, a facial adjustment instruction to close the right eye, close the left eye, open the mouth, frown, smile, etc. Camera 205 then captures a facial image of the presenting person displaying at least one adjusted facial feature, in accordance with the facial adjustment instruction. After image capture, camera 205 transmits the captured facial image displaying the at least one adjusted facial feature to central controller 145. Central controller 145 can access datastore 114 having stored authorized facial images showing at least one adjusted facial feature of the person. In this authentication step, central controller 145 compares the captured image comprising the at least one adjusted facial image, for example an image of the presenting person with a closed left eye, with stored authorized images of adjusted facial features images of the presenting person. In the absence of a match, access is denied and openable gate 215 remains closed in a locked position. When a match can be established between one of the stored authorized facial images displaying the at least one adjusted facial feature and the captured facial image displaying the at least one adjusted facial feature, central controller 145 transmits a signal to unlock electronic lock 230 thus allowing gate 215 to be opened and allowing the person to access work space 115. It is noted that in some embodiments a plurality of facial adjustment instructions to display adjusted facial features may be transmitted, e.g. frown, close left eye, thus resulting in the performance of two or more (i.e. N) second authentication steps. In this case, the second authentication step is performed N times and the second authentication step is successful when the N captured images displaying at least one adjusted facial feature of the presenting person are matched to N stored authorized facial images displaying the at least one adjusted facial feature of the presenting person.

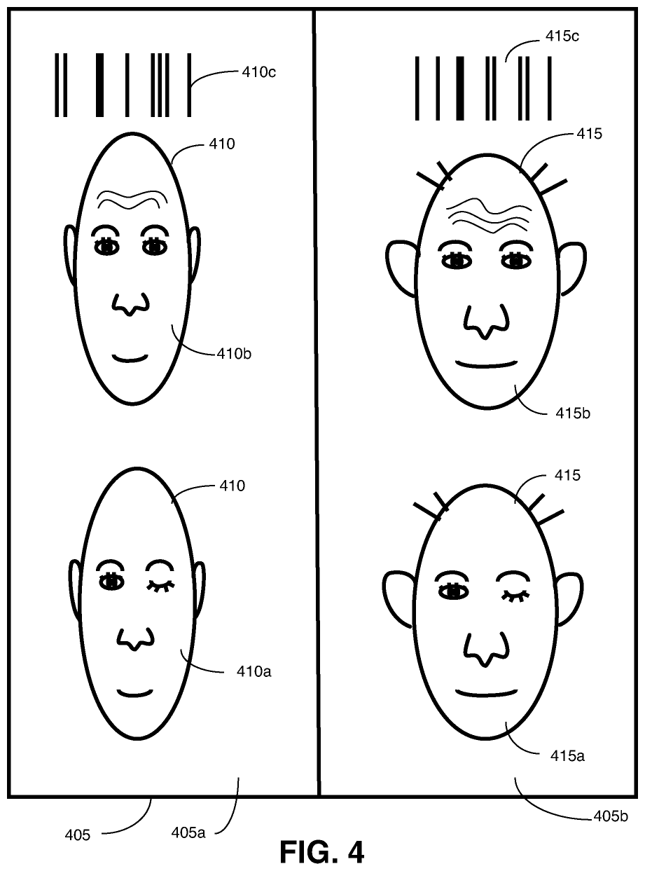

In at least one embodiment, authorized stored facial images comprising adjusted facial features are linked to the first stored authentication token within the datastore 114, as further illustrated in FIG. 4. Shown in FIG. 4 is a schematic overview of a datastore 405 containing authentication information relating to person 410 and person 415. Barcode 410c, representing a stored authorized authentication token corresponding to a first authentication token, is linked to authorized facial images comprising adjusted facial features 410a and 410b representing the adjusted facial features of person 410 (left eye closed, in facial image 410a; and frowning, in facial image 410b). Barcode 410c and authorized facial images 410a and 410b are contained within datastore record 405a. Barcode 415c representing another stored authorized authentication token corresponding to a first authentication token is linked to authorized facial images comprising adjusted facial features 415a and 415b of person 415 (left eye closed in facial image 415a; and frowning in facial image 415b). Barcode 410c and authorized facial images 410a and 410b are contained within datastore record 405b.

Central controller 145 is configured to perform the second authentication step by comparing a received facial image comprising adjusted facial features of person 410 only against stored authorized facial images 410a and 410b comprising adjusted facial features, linked to barcode 410c, and not against the stored authorized facial images 415a and 415b comprising adjusted facial features, linked to barcode 415c, or other stored authorized facial images comprising adjusted facial features (not shown). In an example embodiment, the central controller 145 is configured to perform the second authentication step by comparing a received facial image only against a stored authorized facial image comprising adjusted facial features, where the facial adjustments correspond with the one or more facial adjustment instructions that are provided by the instruction device 210 to the person that is presenting themselves for authorization. Thus, for example, if instruction device 210 has provided a facial adjustment instruction to person 410 to present an adjusted facial feature by closing their left eye, central controller 145 searches authorized facial images 410a and 410b. Central controller 145 then identifies facial image 410a as corresponding with the facial adjustment instruction, and a comparison between the captured image and the authorized facial image comprising adjusted facial features 410a and 410b is only performed using authorized facial image 410a, and not authorized facial image 410b. In this manner, the computer processing capabilities required to perform the second authentication step are substantially reduced relative to an authentication step requiring a comparison against all stored facial images, i.e. those belonging to all authorized persons, comprising adjusted facial features. Furthermore, since central controller 145 is configured to perform the second authentication step so that authentication does not require comparison against all stored images, it is less likely that there will be a security system malfunction due to the inability to correctly resolve the authentication of persons with similar facial features. At the same time, imposters that have misappropriated a first authentication token cannot gain access since they will fail to pass the second authentication step, as they will not have images of the authorized person with various adjusted facial features that correspond to selected facial adjustment instructions that must be performed to obtain certain adjusted facial features for image capture. Similarly, imposters who may present only one facial image of a duly authorized person on a photograph will fail the second authentication step.

In some embodiments, an authentication token can provide permanent access to work space 115. In other embodiments, an authentication token can provide temporary access to work space 115, e.g. for a certain selected week, or daytime access only. In this respect, central controller 145 can be configured so that access to work space 115 is only provided when the first and/or second authentication steps are performed by a person seeking access to work space 115 at an acceptable pre-approved selected time. Conversely, when a person seeks access to work space 115 at a time other than an acceptable pre-approved selected time, access is denied. Accordingly, when the first or second authentication step is performed, the current time of access by the person can be compared to a pre-approved selected time that is stored for this particular person and linked to the barcode for that person. Pre-approved times may be entered for worker 105a and 105b for storage in datastore 114 of the memory component of central controller 145 via input device 140, operated, for example, by a human administrative operator of security system 100. Thus, by way of example, if worker 105a is authorized to access work space 115 in the day time but not at night, while worker 105b is authorized to access work space 115 at any time, when workers 105a and 105b each initiate the authentication process at, e.g. 11:00 PM, security system 100 can deny worker 105a access to work space 115, while providing access to worker 105b. In this manner security system 100 can be configured to temporally control access to work space 115.

Referring again to FIG. 2, in at least one embodiment, authentication module 109 may be configured to include a temperature-detection device 240 coupled to central controller 145. Temperature-detection device 240 is installed and configured to detect the body temperature of a person 410 situated within presentation space 117b. Temperature-detection device 240 may be a temperature-detection device requiring physical contact between a temperature sensor included therein and person 410 within presentation space 117b, for example, by physical contact between a finger of person 410 and a temperature sensor 241. Facial adjustment instructions to person 410 to establish such contact may be provided by instruction device 210. More preferably, however, temperature-detection device 240 is a temperature detection device including temperature sensor 241 that permits remote temperature detection, i.e. a temperature sensor which does not require physical contact between person 410 and temperature sensor 241, such as for example, an infrared temperature scanning device, which may operate at a distance of a few inches from person 410's forehead, or from other sensing zones.

Furthermore, in some embodiments, temperature-detection device 240 may be situated and installed to be fixed in place to allow for temperature detection of person 410 situated within presentation space 117b, for example by fixed attachment to gate support structures 216a or 216b. In other embodiments, the temperature-detection device 240 may be a portable device, including, a hand-held device, which may be operated by another person when person 410 is situated within presentation space 117b.

Temperature-detection device 240 may include any temperature scanner, a body thermometer, or other device for reading the body temperature of a human being, including any temporal temperature scanner, i.e. a temperature scanner detecting body temperature more or less continuously as a function of time. Temperature-detection devices 240 that may be used in accordance herewith include the temperature-detection devices described in U.S. Pat. No. 8,282,274, for example.

In general terms, temperature-detection device 240 can be configured to detect the body temperature of person 410 within presentation space 117b, and subsequently transmit the detected body temperature to central controller 145. Central controller 145 can be configured to transmit a signal to unlock electronic lock 230, thus allowing gate 215 to be opened and allowing person 410 to access work space 115, when the detected body temperature does not deviate from the body temperature of a healthy person, for example, when the body temperature does not exceed an acceptable predefined body temperature of about 37° C., 37.5° C., 38° C., or 38.5° C. Conversely, when the detected body temperature does deviate from the body temperature of a healthy person, and is, for example, in excess of the acceptable predefined body temperature of about 37° C., 37.5° C., 38° C. or 38.5° C., central controller 145 is configured to not transmit a signal to electronic lock 230 so that it may remain locked even if the worker has passed the other security checks. Thus, it is possible to limit access to work space 115 to persons who do not present with elevated body temperatures, for example, and only admit persons to work space persons 115 who present with a body temperature that is in a predefined body temperature range of about 36.5° C. to about 38.5° C. Furthermore, central controller 145 can be configured so that person 410 in presentation space 117b is notified of the detected body temperature via instruction device 210. In the event a person is denied access to work space 115 as a result of a detected abnormality in body temperature, the person may undergo further separate medical examination, as desired. Thus, this example embodiment, may be implemented to control the spread of contagious diseases which cause an increase in the body temperature of person 410 so that their body temperature is not in the predefined body temperature range.

It is noted that in some embodiments, temperature-detection device 240 can be configured to be operable in conditions in which substantial variations in ambient temperatures can occur, for example, due to changing weather conditions. In this respect, temperature-detection device 240 may be configured to correct for variations in ambient temperatures. For example, in the event person 410 presents in cold winter temperatures, temperature-detection device 240, together optionally with central controller 145, may be configured to correct the detected body temperature upwards. Similarly, if a person 410 presents in warm summer temperatures, temperature-detection device 240 together optionally with central controller 145, may be configured to correct the detected body temperature downwards. The foregoing corrections are particularly desirable when the temperature-detection device measures skin surface temperatures.

It is noted that central controller 145 may be configured so that the temperature-detection device 240 can detect the body temperature of person 410 prior to the performance of the authentication step, or during the performance of the first and/or second authentication step, or following the performance of the second authentication step.

In some embodiments, temperature-detection 240 device further may be an infrared sensor configured to detect a heat profile based on the thermal contours of person 410 in presentation space 117b. In such embodiments, central controller 145 can be configured to transmit a signal to unlock electronic lock 230, thus allowing gate 215 to be opened and allowing the person to access work space 115, only when the detected heat profile is consistent with certain characteristics or attributes of an actual person in presentation space 117b. Thus, for example, central controller 145 can be configured so that if person 410 attempts to circumvent authentication by presenting an inanimate object, such as a picture, during an authentication step requiring the presentation of the facial features of person 410, the detection by the temperature detection device 240 of a heat profile which is inconsistent with the presence of person 410, results in the central controller 145 not unlocking electronic lock 230.

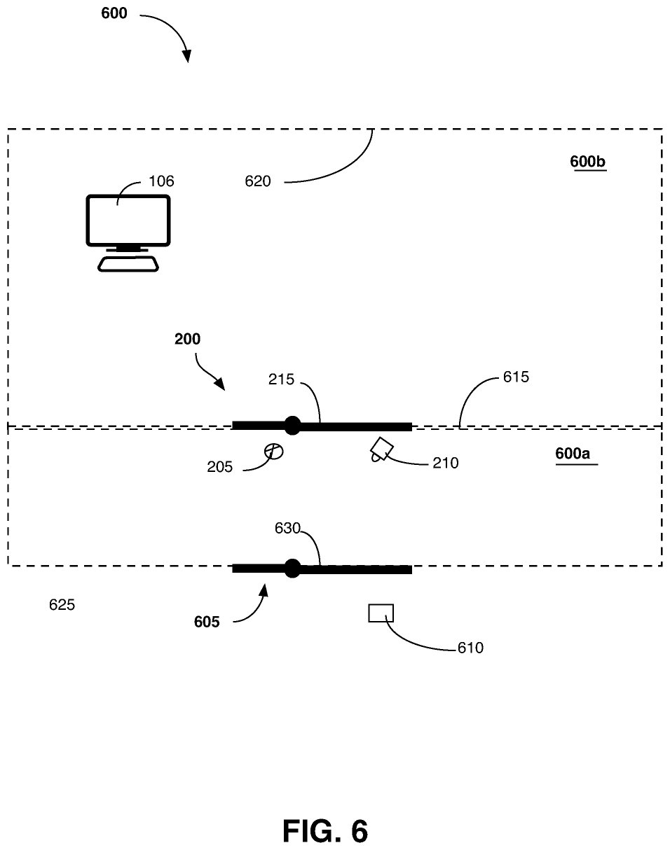

It is noted that in embodiments herein, in which the device receiving the first authentication token and the camera receiving the facial image are separated, these devices may be installed in a manner in which they are spaced away from each other, and can even be located in separate spaces (i.e. separate locations). Thus, the first authentication step may be performed in a first space, and the second authentication step may be performed in a second space, for example a first room and a second room. Access from the first space to the second space may be controlled by another control access device, granting access on completion of the first authentication step. Referring now to FIG. 6, shown therein are spaces 600a and 600b, both separated from exterior 625, and separated from each other by wall 615. In order for a person to access space 600b containing computing device 106 a first authentication step using authentication device 610 is performed while the person is situated in exterior space 625. Upon successful completion of the first authentication step, electronic gate 630 is unlocked via electronic access control device 605 and the person can pass from exterior space 625 into space 600a. In order to access space 600b, a second authentication step is performed, where each of the first and second authentication steps are performed as hereinbefore described. It is noted that in this manner it is possible to contain a person having cleared the first authentication step, but not clearing the second authentication step within space 600a for further inspection.

The present disclosure provides, in another aspect, at least one embodiment of a computer implemented method for unlocking of an electronic access control device of a security system, the method comprising:
capturing, via a camera, a first facial image of a person who is presented to the camera where the camera is positioned in proximity of the electronic access control device;
performing a first authentication step of a two-step authentication process on the person, the first authentication step comprising:
   receiving a first authentication token from the person; and
   authenticating the person using the first authentication token;
performing a second authentication step of the two-step authentication process on the person, the second authentication step comprising:
   selecting one of a plurality of facial adjustment instructions to instruct the person to adjust at least one facial feature when being imaged by the camera;
   prompting an instruction device to transmit the selected facial adjustment instruction to the person;
   capturing, via the camera, a second facial image of the person adjusting the at least one facial feature in accordance with the transmitted facial adjustment instruction;
   receiving at least a portion of the second facial image comprising the adjusted facial feature; and
   authenticating the person when the portion of the second facial image is matched with a corresponding stored authorized image of the person from a datastore of adjusted facial images; and
unlocking the access control device upon successful authentication of the person in the first and second authentication steps.

It should be noted that the method includes receiving at least a portion of the second facial image since depending on the actual facial adjustment instruction to adjust a facial feature only a portion of the image may be needed to capture that adjusted facial feature, such as the upper left quadrant of the person's face when they are instructed to close their eye, for example. Then authentication may only require comparing the portion of the second facial image that is captured with a corresponding stored authorized image of the person from a datastore of adjusted facial images where the corresponding stored authorized facial image includes the same portion of the facial image. This might allow for quicker processing and authentication of the person to access the work space.

In at least one embodiment, the present disclosure provides a method shown in FIG. 5. Thus, referring to FIG. 5 now, the present disclosure includes a method 500 for unlocking an electronic access control device of a security system that leads to a secure space, the method 500 comprising a first step 505 in which a person is presenting themselves to an electronic access control device to seek access to the secured space. It should be noted that there may be an alternative embodiment in which the temperature of the person presenting to the electronic access control device may be checked, as described above, in order to unlock the electronic access control device.

Method 500 further comprises second step 510, which may be automatically initiated or initiated by a person taking an action to request access to the secured space, for example by pushing an installed button, or by using an installed telephone or a mobile telephone to start method 500. When method 500 is initiated, the person presents themselves by positioning themselves in close proximity to the electronic control access device. The electronic control access device can include a gate, which generally will be in a locked position when method 500 is initiated.

Method 500 further comprises a third step 515 comprising capturing a first authentication token, for example, a barcode or biometric features such as a full facial image, or a partial facial image, of the person who is presenting himself. This capture is performed using a camera. The camera is installed in the proximity of a presentation area where the person presents themselves, and generally is in the proximity of the gate.

Method 500 further comprises fourth step 520 comprising authenticating the person in a first authentication step via a central controller. This step is performed by comparing the first authentication token with stored authorized authentication tokens, for example by comparing a captured facial image with stored authorized facial images of the person stored in a datastore. In the event no matching facial image is identified, fifth step 525 is performed and the person is denied access, e.g. by not releasing the locked gate.

In the event that the person is successfully authenticated in the first authentication step, sixth step 530 of method 500 is performed by a central controller. Sixth step 530 comprises selecting a facial feature adjustment instruction from multiple facial adjustment feature instructions. The selected facial adjustment instruction is transmitted to the person using an instruction device 210 as indicated in the seventh step 535. The person responds in accordance with the selected facial adjustment instruction by adjusting at least one of their facial features while the camera captures a facial image of the person with the at least one adjusted facial feature.

Method 500 further comprises eighth step 540 comprising authenticating the person for a second time via a central controller. This step is performed by comparing the captured adjusted facial image with stored authorized adjusted facial images of the person stored in a datastore. In the event no matching stored authorized adjusted facial image is identified, fifth step 525 is performed and the person is denied access, e.g. by not releasing the locked gate. In the event a stored authorized adjusted facial image is identified that matches the captured adjusted facial image, ninth step 545 of method 500 is performed and the electronic access protection device provides the person with access to the secured space, e.g. by unlocking the gate. Method 500 can then be repeated when another person presents himself to the electronic control access device.

It should be noted that while various functions have been described as being performed by a central controller, in at least one embodiment, these functions can be performed by another computing device which may be local to the electronic gate.

While the applicant's teachings described herein are in conjunction with various implementations or embodiments for illustrative purposes, it is not intended that the applicant's teachings be limited to such implementations. On the contrary, the applicant's teachings described and illustrated herein encompass various alternatives, modifications, and equivalents, without departing from the implementations or embodiments described herein, the general scope of which is defined in the appended claims.

1. A security system comprising:
an electronically lockable access control device configured to be unlocked upon authentication of a person presenting to the access control device; and
an authentication module coupled to the access control device, the authentication module comprising:
an instruction device;
a camera configured to capture a first facial image of at least a portion of the face of the person presenting to the camera; and
a central controller comprising a processor and a memory that is accessible by the processor, the central controller being communicatively coupled to the instruction device and the camera, and the memory having program instructions stored thereon, that when executed by the processor, configure the central controller to:
perform a first authentication step of a two-step authentication process on the person, the first authentication step comprising:
receiving a first authentication token from the person and authenticating the first authentication token; and
perform a second authentication step of the two-step authentication process on the person, the second authentication step comprising:
selecting one of a plurality of facial adjustment instructions to instruct the person to adjust at least one facial feature when being imaged by the camera;
sending the selected facial adjustment instruction to the instruction device;
providing the selected facial adjustment instruction via the instruction device to the person;
capturing, via the camera, a second facial image of the person while the person is adjusting the at least one facial feature in accordance with the transmitted facial adjustment instruction;
receiving, at the central controller, at least a portion of the second facial image comprising the at least one adjusted facial feature of the person; and
authenticating the person when the portion of the second facial image is matched with a corresponding stored authorized adjusted facial image of the person obtained from a datastore of adjusted facial images of the person; and
unlock the access control device when there is successful authentication in the first and second authentication steps.

2. A security system according to claim 1, wherein the second authentication step is performed only when there is successful authentication in the first step.

3. A security system according to claim 1, wherein the camera is configured to capture and receive the first authentication token.

4. A security system according to claim 1, wherein the authentication module comprises an additional device that is configured to receive the first authentication token wherein the additional device is a device other than the camera.

5. A security system according to claim 1, wherein the central controller is in communication with a datastore comprising a plurality of stored authorized authentication tokens and the first authentication step comprises performing a matching between the received authentication token and the stored authorized authentication tokens, where each stored authorized authentication token is linked to stored authorized facial images comprising adjusted facial features of the person, and the central controller is configured to perform the authenticating in the second authentication step by performing solely a matching between the captured adjusted facial image and one of the stored authorized facial images that are linked to the first authentication token and comprise the adjusted facial features of the person.

6. A security system according to claim 5, wherein at least one of the stored authorized facial images that corresponds with the captured adjusted facial feature image has one or more facial adjustments that corresponds with one or more facial adjustments given in the provided facial adjustment instruction.

7. A security system according to claim 1, wherein the first authentication token comprises a 1D or 2D barcode.

8. A security system according to claim 1, wherein the first authentication token comprises the first facial image captured by the camera, and the authentication comprises performing a matching between the captured first facial image against a datastore comprising stored authorized facial images.

9. A security system according to claim 1, wherein the camera or the instruction device is situated in close proximity to the electronically lockable access control device.

10. A security system according to claim 1, wherein the instruction device is configured to provide visual instructions or audible instructions to the person.

11. A security system according to claim 10, wherein the visual instructions comprise a cartoon representing an adjusted facial feature or text instructions for the person to adjust at least one of their facial features.

12. (canceled)

13. A security system according to claim 1, wherein the central controller is configured to perform the first and the second authentication steps in different first and second spaces, respectively.

14. A security system according to claim 13, wherein the electronic access control device comprises first and second electronic access control components, the first electronic access control component being unlocked upon successful authentication in the first authentication step, and the second electronic access control component being unlocked upon successful authentication in the second authentication step.

15. A security system according to claim 1, wherein the central controller is configured to unlock the access control device only when the first and/or second authorization step is also performed at a selected pre-approved time.

16. A security system according to claim 1, wherein the electronic access control device further includes a temperature-detection device to detect the body temperature of the person, the temperature-detection device being coupled to the central controller, the central controller being configured to unlock the access control device when the detected body temperature of the person is within a predefined body temperature range.

17. A security system according to claim 16, wherein the temperature-detection device is configured to detect the body temperature of the person following the performance of the first and second authentication step.

18. A security system according to claim 16, wherein the predefined body temperature range is from about 36.5° C. to about 38.5° C.

19. A computer implemented method for unlocking of an electronic access control device of a security system, the method comprising:
capturing, via a camera, a first facial image of a person presented to the camera where the camera is positioned in proximity of the electronic access control device;
performing a first authentication step of a two-step authentication process on the person, the first authentication step comprising:
receiving a first authentication token from the person; and
authenticating the person using the first authentication token;
performing a second authentication step of the two-step authentication process on the person, the second authentication step comprising:
selecting one of a plurality of facial adjustment instructions to instruct the person to adjust at least one facial feature when being imaged by the camera;
prompting an instruction device to provide the selected facial adjustment instruction to the person;
capturing, via the camera, a second facial image of the person adjusting the at least one facial feature in accordance with the provided facial adjustment instruction;
receiving at least a portion of the second facial image comprising the adjusted facial feature; and
authenticating the person when the portion of the second facial image is matched with a corresponding stored authorized image portion of the person from a datastore of stored authorized adjusted facial images; and
unlocking the access control device upon successful authentication of the person in the first and second authentication steps.

20.-22. (canceled)

23. A method according to claim 19, wherein the first authentication step comprises performing a matching between the received authentication token and stored authorized authentication tokens, where each stored authorized authentication token is linked to stored facial images comprising adjusted facial features of the person, and the authenticating in the second authentication step is performed solely based on a matching between the captured adjusted facial image and one of the stored authorized facial images that are linked to the first authentication token and comprise adjusted facial features of the person.

24.-33. (canceled)

34. A method according to claim 19, wherein the electronic access control device further includes a temperature-detection device and the method further comprises detecting the body temperature of the person with the temperature-detection device and unlocking the access control device when the detected body temperature of the person is within a predefined body temperature range.

35.-36. (canceled)
